(19) United States
(12) Patent Application Publication
(10) Pub. No.: US 2008/0118600 A1
(43) Pub. Date: May 22, 2008

(54) Means for Improving Fermenting Capacities of Active Yeasts and Uses Thereof

(75) Inventors: Jean-Michel Salmon, Castelnau-le-Lez (FR); Anne Ortiz-Julien, Gagnac Sur Garonne (FR)

Correspondence Address: NIXON & VANDERHYE, PC, 901 NORTH GLEBE ROAD, 11TH FLOOR, ARLINGTON, VA, 22203, US

(73) Assignees: LALLEMAND SAS, Blagnac Cedex (FR); INSTITUT NATIONAL DE LA RECHERCHE AGRONOMIQUE (INR, Paris Cedex (FR)

(21) Appl. No.: 11/791,017

(22) PCT Filed: Nov. 21, 2005
(86) PCT No.: PCT/FR05/02887

§ 371 Date: Jun. 11, 2007

(30) Foreign Application Priority Data
Nov. 19, 2004 (FR) 0412309

(51) Int. Cl. C12C 11/00; A23L 1/28; C12G 3/00; C12N 1/00; C12G 1/00

(52) U.S. Cl. 426/11; 426/62; 435/255.1; 435/255.21

(57) ABSTRACT

The invention concerns dry active yeasts useful for rehydration and/or alcoholic fermentation, or rehydrated active yeasts useful for fermentation, or yeasts useful for fermentation, having a sterol content exhibiting at least two conjugated double bonds at the B cycle of at least 900 μg/g of dry weight. The invention is useful for producing fermented alcoholic beverages.

The invention relates to means for improving the fermenting capacities of active yeasts. The invention more particularly relates to dry active yeasts (abbreviated to DAY) or rehydrated active yeasts, with a high sterol content, and to the production thereof. The invention also relates to the uses of these yeasts in alcoholic fermentation processes, in particular for the manufacture of fermented alcoholic beverages.

Sterols are important constituents of the cell membrane of yeasts. The sterol concentration should be optimal to ensure the integrity of the membrane, which is a guarantee of activity of the membrane proteins, such as permeases.

Under fermenting conditions, if some of these permeases no longer function, a drop in cell viability is observed, which may result in the death of the cell.

The sterol composition of yeasts thus plays a fundamental role in their fermenting capacity.

To increase the sterol concentration of yeasts, it has been proposed to incorporate exogenous sterols during the growth of yeasts. However, their insoluble nature in aqueous medium is a limiting factor.

To overcome this drawback, it has been recommended to use surfactants to dissolve them, which limits the possibilities of direct addition of sterols to the culture medium.

In the case of deficiencies of lipid factors, the addition of fine must deposits to the fermentation must has been recommended in order to supply the medium with nutrients such as sterols and unsaturated fatty acids in complex form. However, such an addition is not always easy to perform in cellars.

The inventors have found that these drawbacks can be overcome by using inactivated yeasts with a high content of certain types of sterols, either naturally, or by enrichment, or by using sterol compositions in soluble form. It turned out, effectively, that the addition of such yeasts or compositions to the medium for rehydrating active dry yeasts or to active yeasts in the fermentation must makes it possible to stabilize the sterols and to have soluble forms that can be efficiently incorporated by the yeasts.

Surprisingly, the fermenting capacities of the active yeasts of the fermentation medium are particularly high, especially during the stationary phase of fermentation, the duration of the fermentation generally being shorter.

One subject of the invention is thus dry active yeasts or rehydrated active yeasts with improved fermenting capacities. The invention is also directed toward a process for obtaining them that is easy to implement.

The invention is also directed toward exploiting the fermenting properties of in these active yeasts for the manufacture of alcoholic fermented beverages, in particular wines, beers and spirits such as vodka or whiskey.

The invention is thus directed toward DAYs that can be used in rehydration and/or alcoholic in fermentation, or rehydrated active yeasts that can be used in fermentation, or yeasts that can be used in fermentation, with a content of sterols containing at least two conjugated double bonds in ring B of at least 200 μg/g of dry weight and more especially of at least 900 μg/g of dry weight.

The term “yeasts that can be used in fermentation” means yeast cream obtained from their production or frozen yeasts.

They are more especially winemaking yeasts or brewing yeasts, or alternatively distillery yeasts, which includes yeasts of the Saccharomyces genus or of the non-Saccharomyces genus. Mention will be made in particular of Saccharomyces belonging to the species cerevisiae, uvarum and bayanus.

The sterol enrichment of DAYs is advantageously obtained by incorporating sterol compositions in soluble form using micelles.

Advantageously, these micelles comprise at least 10 to 20 mg/g (dry weight) of sterols, in particular at least 15 mg/g (dry weight), and about 600 to 800 mg/g (dry weight) of lipopolysaccharide-protein complexes, which can be precipitated with 80% (v/v) ethanol, in particular 660 to 670 mg/g (dry weight), with an average molecular weight of 160 to 170 kDa approximately.

Sterols comprise at least 20-25% of ergosterol and 20-25% of zymosterol, other sterols being present, for instance fecosterol, lanosterol or ergosta-5,7-dienols, without this list being limiting.

A composition of such micelles is given in the examples.

According to one embodiment of the invention, the sterol-enriched DAYs are obtained by adding to their rehydration medium or to the fermentation must inactivated yeasts, which themselves have a content of sterols containing at least two conjugated double bonds in ring B of at least 200 μg/g of dry weight and more especially of at least 900 μg/g (dry weight).

Such inactivated yeasts are yeasts that are naturally rich in sterols or yeasts that are enriched in sterols, especially in ergosterol and/or zymosterol and/or ergosta-5,7-dienols, advantageously according to the techniques known to those skilled in the art.

Preferably, the yeasts used advantageously have a content of sterols containing two conjugated double bonds in ring B of 900 to 1250 μg/g of dry weight.

The amounts of inactivated yeasts added to the rehydration medium are from about 20 to 500 g/l, preferably from 100 to 200 g/l and especially about 150 g/l.

The enrichment of the DAYs is performed by means of transferring the sterols in the form of micelles using inactivated yeasts.

According to another embodiment of the invention, the sterol enrichment of the yeasts is obtained by adding, to DAYs in their rehydration medium or to DAYs added to fermentation musts, a lipid composition containing a sterol fraction of sterols comprising ergosterol and zymosterol and a lipopolysaccharide-protein complex that can be precipitated with ethanol.

These compositions advantageously contain from 10% to 70% by dry weight of ergosterol and from 10% to 70% by dry weight of zymosterol, in a proportion of from 10 to 20 mg of total sterols per g and from 500 to 1000 mg/g of lipopolysaccharide-protein complex.

Advantageously, the sterol fraction also comprises one or more sterols such as fecosterol, lanosterol, ergosta-5,7-dienols (dihydroergosterol, methyl-zymosterol, demethyllanosterol and ergosta-7,22-dien-3-ol).

The lipid compositions as used in the above process are novel products and, in this respect, are also included in the field of the invention.

These lipid compositions especially have the following physicochemical properties:
they correspond to an amphiphilic colloidal form with no true solubility in suspension in pure water at 4° C.,
they cannot cross filtration membranes with a cutoff threshold of 0.2 μm
they have a surfactant effect that is manifested only above a concentration of 0.5 g/l of dry weight in aqueous phase,
they have a property of self-aggregating above a concentration of 0.05 g/l of dry weight in aqueous phase to form micellar aggregates with a mean diameter of about 380 nm (value obtained by measuring the laser light scattering) via the PCS (Photo Correlation Spectroscopy) technique.

To obtain a maximum surfactant effect for the sterols, the above compositions are used in a proportion of from 1.5 to 3 g/l and preferably of 2 g/l of rehydration medium or from 20 to 100 g/hecto of fermentation must.

Yeasts with a high sterol content, as obtained by enrichment during their rehydration or fermentation phase, by means of the transfer and incorporation of the sterols in the form of micelles using inactivated yeasts, or by incorporating said lipid compositions, are of great interest in fermentation processes, taking into account their high fermenting capacities during the stationary phase of fermentations.

In addition, this high sterol content reduces the production of volatile acids, which makes it possible to improve the organoleptic properties of the fermentation products.

The invention is also directed toward the use, as technological auxiliaries, of sterol-rich inactivated yeasts, as defined above, in the rehydration media of DAYs or in alcoholic fermentation media, for the production of alcoholic fermented beverages, in particular wines or beers, or alcohols of the type such as whiskey, vodka and the like.

The invention is also directed toward a process for producing such beverages, comprising the addition of sterol-enriched dry active yeasts as defined above, in a proportion of from 100 to 200 g/l and preferably 150 g/l of fermentation medium. These yeasts are either yeasts, which may or may not be rehydrated, which are naturally rich in sterols, or yeasts that are enriched as defined above, i.e. rehydrated active yeasts or DAYS.

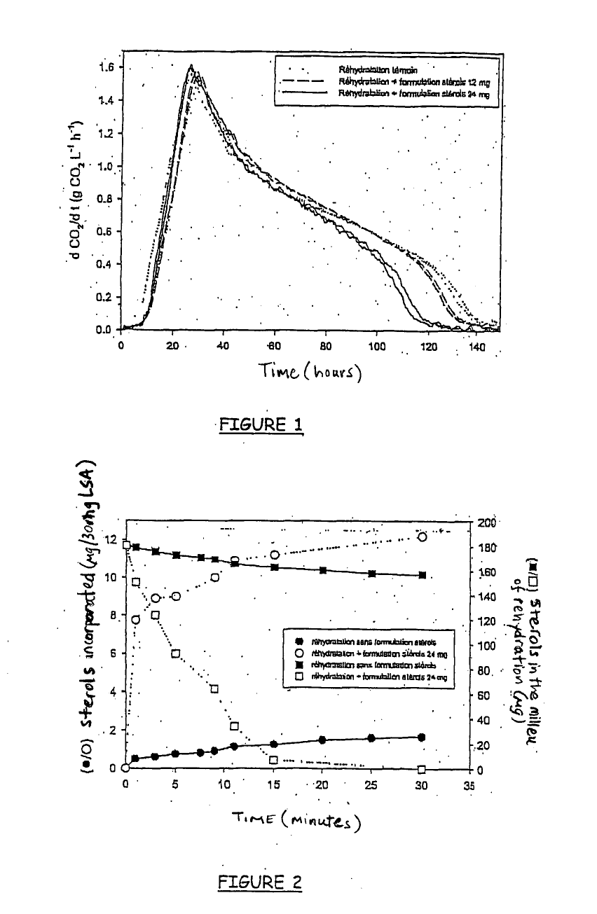

Other characteristics and advantages of the invention are given in the examples that follow. In these examples, reference will be made to FIGS. 1 and 2, which show, respectively:

FIG. 1—the rehydration of a DAY in the presence of sterol preparations according to the invention, at various sterol contents; and

FIG. 2—the incorporation of sterols by DAYs during a rehydration phase using a sterol preparation according to the invention.

### EXAMPLE 1

### Formulation of Stabilized Sterols in Soluble Form

10 ml of suspension in water of a sample of inactivated yeasts, at a concentration of 150 g/l of dry weight, are subjected to an incubation step, with stirring, at 37° C. for 30 minutes.

By centrifugation of the preparation at 11 300×g for minutes, a supernatant 1 and a pellet 1 are obtained. The supernatant 1 is subjected to an ultracentrifugation step at 85 000×g for 3 hours at 4° C. A pellet 2, a supernatant 2 and a white surface colloid are obtained. The white colloidal fraction is freeze-dried to give the stabilized sterol preparation in soluble form (about 20 mg).

### EXAMPLE 2

### Effect of the Sterol Formulation According to Example 1 on the Fermentation Profile of a Synthetic Must

1 g of Lalvin EC1118® dry active yeast is rehydrated in 10 ml of a rehydration medium consisting of water supplemented with 50 g/l of glucose. The rehydrations are performed at 37° C. for 30 minutes, in the presence of 12 or 24 mg of the sterol formulation of Example 1 and, as a control, in the absence of sterol formulation.

Two fermentations were performed in parallel and in duplicate on a synthetic must MS300-stigmasterol corresponding to a nitrogen-rich must (300 mg of nitrogen/l), deficient in sterols and containing 200 g/l of fermentable sugars.

Four fermenters containing 1.1 l of synthetic must MS300-stigmasterol are inoculated with 2.2 ml of rehydration solution. The temperature is maintained at 24° C. The production of CO₂ is recorded as a function of time.

The results obtained are given in FIG. 1. Examination of this figure shows that the fermentations performed by the rehydrated yeasts in the presence of used sterol formulation end before the control fermentations.

Globally, the kinetics observed for the fermentations performed by the rehydrated yeasts in the presence of the stabilized sterol formulation in soluble form are faster once the stationary phase has begun.

### EXAMPLE 3

### Effect of the Sterol Formulation According to Example 1 on the Activation of the Incorporation of Sterols by the DAYs

The process is performed as in Example 1, but the rehydration is performed in the presence of 2 μl of [4-¹⁴C] cholesterol (50-60 mCi/mMol (Amersham CFA 128)), dissolved in 20 mμl of tergitol/ethanol (v/v) mixture in the presence or absence of 24 mg of the sterol formulation of Example 1 for the manufacture of alcoholic fermented beverages.

The radioactivity incorporated over time by the DAYs is measured by proceeding as follows:

300 μl of medium are filtered under partial vacuum on a Whatman GF/C glass fiber filter, which is rinsed with 3 ml of distilled water at 4° C., dried and counted by liquid scintillation on a Beckman LS 6500 counter, after addition of 20 ml of Fluoransafe 2® (BDH) scintillant liquid.

The results obtained are given in FIG. 2. It is found that during rehydration, the presence of the sterol formulation according to the invention allows a more efficient incorporation of the sterols of the medium by the DAYS.

### EXAMPLE 4

### Sterol Enrichment of Inactivated Yeasts

The yeast strains are cultured under a constant and high aeration. The propagation temperature is from about 15 to 40° C. and preferably from 25 to 35° C.

The yeasts are cultured by constant supply of carbon sources and nitrogen sources, and also by an intermittent supply of essential growth factors. The carbon sources are generally derived from molasses and may be sugar or beetroot molasses or a mixture thereof in variable concentrations. The nitrogen source may originate from various ammoniacal derivatives, for instance ammonium hydroxide, ammonium chloride, ammonium sulfate, ammonium phosphate, diammonium phosphate or crude protein extracts.

The yeast propagation is performed according to a program comprising growth rate variations. The maximum growth rates are from 0.05/h to 0.25/h and preferably from 0.15 to 0.2/h.

The sterol enrichment of the yeast may be performed by specifically modifying the carbon and nitrogen sources so as to reduce the total level of nitrogen relative to that of carbon.

It is also possible to add specific sterol precursors in a controlled manner. Preferred sources of sterol precursors include ethanol, acetic acid, squalene and lanosterol. These precursors may be added in purified or crude form.

### EXAMPLE 5

### Lipid Compositions Used as Additives in Yeast Rehydration Media and/or in Fermentation Musts

These compositions are obtained via a process comprising the steps:
of incubation with stirring of a suspension in water of inactivated winemaking yeasts under conditions that allow the production of micelles formed from fractions specifically enriched in sterols incorporated into lipid-protein complexes,
of separation of the micelle fractions, followed by freeze-drying.

The separation step, after the incubation, of the micelle fractions is more especially performed by centrifugation. The collected supernatant is then subjected to an ultracentrifugation step to increase the sterol enrichment of the micelle fraction, and the supernatant colloidal fraction is isolated and freeze-dried.

The compositions obtained contain a sterol fraction composed of 10% to 70% and especially from 20% to 25% by dry weight of ergosterol, from 10% to 70% and especially from 20% to 25% by dry weight of zymosterol in a proportion of 10 to 20 mg of total sterols per g, and a lipopolysaccharide-protein complex that can be precipitated with 80% (v/v) ethanol, with an average molecular weight of about 160-170 kDa, in a proportion of 600 to 800 mg/g.

The sterol fraction also comprises one or more sterols, such as fecosterol, lanosterol and ergosta-5,7-dienols.

In the lipopolysaccharide-protein complex, the polysaccharides, the lipids and the proteins are advantageously present in a proportion of:
20% to 34% and especially 27% for the poly-saccharides,
55% to 70% and especially 61% for the lipids, and
10% to 15% and especially 12% for the proteins.

Preferred polysaccharides have an average MW of about 30-35 kDa and in particular from 30 to 32 kDa and contain mannose, glucose and inositol. Suitable respective proportions correspond to:
55% to 70% and especially 61% for mannose,
30% to 40% and especially 33% for glucose,
0.5% to 1% and especially 0.9% for inositol.

The lipid fraction comprises, in variable amounts, preferably C17:1, C16:0 and C18:0 fatty acids. This lipid fraction is associated with the polysaccharides via a phosphatidylinositol group.

The remainder of the preparation is formed from membrane fragments that are soluble in 80% (v/v) ethanol.

The micelles formed are composed, for example, of:
1) at least 15 mg/g (dry weight) of sterols, composed of at least 20-25% ergosterol, 20-25% zymosterol and variable amounts of other sterols (fecosterol, lanosterol, ergosta-5,7-dienols, etc.),
2) 667 mg/g (dry weight) of lipopolysaccharide-protein complexes, which can be precipitated with 80% (v/v) ethanol, with an average molecular weight of 167 kDa, composed of 27% polysaccharides, 61% lipids and 12% proteins by weight. The polysaccharides have an average molecular weight of 31.7 kDa, and contain 61% mannose, 33% glucose and 0.9% inositol. The lipid fraction is formed from C17:1, C16:0 and C18:0 fatty acids in variable amounts. The lipid fraction is associated with the polysaccharides by means of a phosphatidylinositol anchor,
3) 333 mg/g (dry weight) of various substances that are soluble in 80% (v/v) ethanol.

1. Dry active yeasts that can be used in rehydration and/or in alcoholic fermentation, or rehydrated active yeasts that can be used in fermentation, or yeasts that can be used in fermentation, with a content of sterols containing at least two conjugated double bonds in ring B of at least 200 μg/g of dry weight.

2. The dry active yeasts as claimed in claim 1, characterized in that they are winemaking yeasts or brewing yeasts or alternatively distillery yeasts, including yeasts of the Saccharomyces genus or of the non-Saccharomyces genus.

3. The dry active yeasts as claimed in claim 2, characterized in that they belong to the Saccharomyces genus, in particular to the species cerevisiae, uvarum and bayanus.

4. A process for obtaining active yeasts as claimed in claim 1, characterized by the enrichment in sterols of the dry active yeasts by incorporating sterol compositions in soluble form using micelles.

5. The process as claimed in claim 4, characterized in that the sterol-enriched DAYs are obtained by ration addition to their rehydration medium or to the fermentation must of inactivated yeasts, which themselves have a content of sterols containing at least two conjugated double bonds in ring B of at least 200 μg/g of dry weight and more especially of at least 900 μg/g (dry weight).

6. The process as claimed in claim 4, characterized in that said inactivated yeasts are yeasts that are naturally rich in sterols or yeasts that are enriched in sterols, especially in ergosterol and/or zymosterol and/or ergosta-5,7-dienols.

7. The process as claimed in claim 5, characterized in that said yeasts have a content of sterols containing two conjugated double bonds in ring B of 900 to 1250 μg/g (dry weight).

8. The process as claimed in claim 5, characterized in that the amounts of inactivated yeasts added to the rehydration medium are from about 20 to 500 g/l.

9. The process as claimed in claim 4, characterized in that the sterol enrichment of the dry active yeasts is performed by adding, to their rehydration medium or to DAYs of fermentation musts, a lipid composition containing:
a fraction of sterols of comprising ergosterol and zymosterol, and
a lipopolysaccharide-protein complex that can be precipitated with ethanol.

10. The process as claimed in claim 9, characterized in that the lipid compositions contain from 10% to 70% by dry weight of ergosterol and from 10% to 70% by dry weight of zymosterol, in a proportion of from 10 to 20 mg of total sterols per g and from 500 to 1000 mg/g of lipopolysaccharide-protein complex.

11. The process as claimed in claim 9, characterized in that the sterol fraction also comprises one or more sterols such as fecosterol, lanosterol and ergosta-5,7-dienols.

12. The process as claimed in claim 9, characterized by the addition of said compositions, in a proportion of from 1.5 to 3 g/l and preferably 2 g/l of rehydration medium or from 20 to 100 g/hl of fermentation must.

13. The use, as technological auxiliaries, of sterol-rich inactivated yeasts, as used in the process of claim 5, in rehydration media of DAYs or in alcoholic fermentation media, for the purposes of producing a beverage by alcoholic fermentation.

14. A process for producing alcoholic fermented beverages, characterized in that it comprises the use of at least one dry active yeast having a content of sterols, or of a rehydrated active yeast, as claimed in claim 1, or as obtained via the enrichment in sterols of dry active yeasts by incorporating sterol compositions in soluble form using micelles.

15. The process as claimed in claim 14, characterized in that it comprises the addition of said yeast(s) in a proportion of from 100 to 200 g/l and preferably 150 g/l of fermentation medium.

16. The process of claim 14, wherein the alcoholic beverage is selected from wine, beer, whiskey and vodka.
